SEQUENCE LISTING (17 sequences)

SEQ ID NO: 1 - DNA, 52 bp, synthetic construct
  source 1..52: mol_type = other DNA
cacccccgaaagagtttgagtggaactcgagttccactcaaactctttcggg

SEQ ID NO: 2 - DNA, 52 bp, synthetic construct
  source 1..52: mol_type = other DNA
aaaacccgaaagagtttgagtggaactcgagttccactcaaactctttcggg

SEQ ID NO: 3 - DNA, 52 bp, synthetic construct
  source 1..52: mol_type = other DNA
caccgcagacttgcagtttaagtatctcgagatacttaaactgcaagtctgc

SEQ ID NO: 4 - DNA, 52 bp, synthetic construct
  source 1..52: mol_type = other DNA
aaaagcagacttgcagtttaagtatctcgagatacttaaactgcaagtctgc

SEQ ID NO: 5 - DNA, 52 bp, synthetic construct
  source 1..52: mol_type = other DNA
caccgcaaggaaataaagtttcagtctcgagactgaaactttatttccttgc

SEQ ID NO: 6 - DNA, 52 bp, synthetic construct
  source 1..52: mol_type = other DNA
aaaagcaaggaaataaagtttcagtctcgagactgaaactttatttccttgc

SEQ ID NO: 7 - DNA, 52 bp, synthetic construct
  source 1..52: mol_type = other DNA
cacctcacgaattgcccgaacattactcgagtaatgttcgggcaattcgtga

SEQ ID NO: 8 - DNA, 52 bp, synthetic construct
  source 1..52: mol_type = other DNA
aaaatcacgaattgcccgaacattactcgagtaatgttcgggcaattcgtga

SEQ ID NO: 9 - DNA, 52 bp, synthetic construct
  source 1..52: mol_type = other DNA
cacccccattaggtgcccatatttactcgagtaaatatgggcacctaatggg

SEQ ID NO: 10 - DNA, 52 bp, synthetic construct
  source 1..52: mol_type = other DNA
aaaacccattaggtgcccatatttactcgagtaaatatgggcacctaatggg

SEQ ID NO: 11 - DNA, 21 bp, synthetic construct
  source 1..21: mol_type = other DNA
tgtgggcatcaatggatttgg

SEQ ID NO: 12 - DNA, 22 bp, synthetic construct
  source 1..22: mol_type = other DNA
acaccatgtattccgggtcaat

SEQ ID NO: 13 - DNA, 19 bp, synthetic construct
  source 1..19: mol_type = other DNA
accgggcatagtggttgga

SEQ ID NO: 14 - DNA, 22 bp, synthetic construct
  source 1..22: mol_type = other DNA
atggtacacggttctcaacatc

SEQ ID NO: 15 - DNA, 16 bp, synthetic construct
  source 1..16: mol_type = other DNA
gtccagttttcccagg

SEQ ID NO: 16 - DNA, 16 bp, synthetic construct
  source 1..16: mol_type = other DNA
gcgagcacagaattaa

SEQ ID NO: 17 - DNA, 21 bp, synthetic construct
  source 1..21: mol_type = other DNA
ttggctattgggaacgtcctt

(19) United States
(12) Patent Application Publication
(10) Pub. No.: US 2026/0146232 A1
(43) Pub. Date: May 28, 2026

(54) METHODS FOR PROMOTING HOMING AND ENGRAFTMENT OF HEMATOPOIETIC STEM CELLS

(71) Applicant: The Trustees of Indiana University, Bloomington, IN (US)

(72) Inventors: Reuben Kapur, Zionsville, IN (US); Zuepeng Wang, Indianapolis, IN (US); Broxmeyer Hal, Bloomington, IN (US)

(21) Appl. No.: 19/122,522

(22) PCT Filed: Oct. 20, 2023
(86) PCT No.: PCT/US2023/077413

§ 371 Date: Apr. 18, 2025

Related U.S. Application Data
(60) Provisional application No. 63/380,393, filed on Oct. 21, 2022.

(51) Int. Cl. C12N 5/0789; C12N 15/85

(52) U.S. Cl. CPC C12N 5/0647; C12N 15/85; C12N 2501/32; C12N 2501/40

(57) ABSTRACT

Methods to improve homing and engraftment of hematopoietic stem cells, particularly cord blood CD34+ cells, for use in hematopoietic cell transplantation through the regulation of expression of YTHDF2 or FTO in CD34+ cells. The methods include transiently repressing expression of YTHDF2 through exposure of the CD34+ cells to YTHDF2 repressor compound. The methods also include exposing CD34+ cells to a FTO expression activator compound to transiently increase FTO expression in the cells.

### CROSS-REFERENCE TO RELATED APPLICATION

The present application claims priority to U.S. Provisional Application No. 63/380,393, filed on Oct. 21, 2022, and entitled “METHODS FOR PROMOTING HOMING AND ENGRAFTMENT OF HEMATOPOIETIC STEM CELLS,” the entire disclosure of which is expressly incorporated by reference herein.

### FIELD OF THE INVENTION

This invention relates generally to the field of hematopoietic cell transplantation. More particularly, the invention concerns methods to improve homing and engraftment of intravenously administered stem cells.

### BACKGROUND

Hematopoietic stem cells (HSCs) are the only cells that give rise to all blood cell types and are critical for successful hematopoietic cell transplantation (HCT). (Doulatov, et al., Cell Stem Cell 2012 Feb. 3; 10(2): 120-136.) Cord blood (CB) has been used to treat patients with both malignant and non-malignant haematological disorders over the last 30 years. (Copelan, EA, N Engl J Med 2006 Apr. 27; 354(17):1813-1826). HCT procedures require the HSCs to be harvested from cord blood, healthy normal donors or from patients. The harvested HSCs are then subsequently administered to patients whose hematopoietic system and presumably disease tissue has been eradicated. After transplantation, the HSCs travel to or “home” to the appropriate bone marrow micro-environmental niches. Once lodged within the appropriate niches, also referred to as engraftment, these cells proliferate and produce new HSC, a process called self-renewal. The cells also differentiate into lineage restricted progenitor cells and mature cells, thereby restoring the blood forming hematopoietic system in the patient.

However, the limited number of HSCs in a single unit of CB greatly impacts the usage of CB for HCT as successful transplantation procedures require a sufficient number of cells to be administered to the patient. Although several efforts have been devoted to ex vivo produce HSCs and enhance CB HCT, development of newer strategies to improve single-CB-unit HCT by enhancing the homing of HSCs, and thus the engraftment efficiency of HSCs is critical.

When intravenously transplanted HSCs home to the bone marrow (BM) and implant in microenvironmental niches, stromal cell-derived factor-1 (SDF-1)/CXCR4 chemotactic axis plays a major role in this process. (Peled, et al., Science 1999 Feb. 5; 283(5403): 845-848; Chute J P, Curr Opin Hematol 2006 November; 13(6):399-406). Prior work on the regulation of the (SDF-1)/CXCR4 chemotactic axis has improved migration and homing of HSCs from peripheral blood (PB) to BM. For example, prostaglandin E2 (PGE2) enhances HSC homing by facilitating HSC chemotaxis toward SDF-1 gradients through upregulation of CXCR4 cell surface expression (U.S. Pat. No. 11,459,545). Mild heat exposure promotes incorporation of CXCR4 into lipid rafts, thus enhancing HSC chemotaxis and engraftment (Capitano, et al., Stem Cells 2015 June; 33(6):1975-1984). While these approaches have enhanced HSC homing, further development of better and safer methods that can enhance homing and engraftment of HSCs is still needed. Aspects of the invention disclosed herein address this need.

### SUMMARY OF THE INVENTION

Some aspects of the present invention provided methods of treating hematopoietic stem cells, comprising the steps of providing at least one compound that contacts the hematopoietic stem cells then removing the at least one compound from contact with the hematopoietic stem cells prior to transplantation of the hematopoietic stem cells into a subject.

A first aspect of the invention includes improved homing and engraftment of hematopoietic stem cells by enhancing expression of CXCR4 through regulating expression of YTHDF2 or FTO.

A second aspect of the invention includes improved homing and engraftment of hematopoietic stem cells by transiently repressing expression of YTHDF2 in HSCs.

A third aspect of the invention includes improved homing and engraftment of hematopoietic stem cells by transiently increasing expression of FTO in HSCs.

A first embodiment is a method of preparing hematopoietic stem cells for homing during stem cell transplantation, comprising contacting hematopoietic stem cells with a YTHDF2 repressor compound for about 8 to about 48 hours; and removing the YTHDF2 repressor compound from contact with the hematopoietic stem cells prior to transplantation of the hematopoietic stem cells into a subject.

A second embodiment is a method wherein the hematopoietic stem cells are harvested from embryonic cord blood.

A third embodiment is a method wherein the YTHDF2 repressor compound is Raloxifene.

A fourth embodiment is a method wherein the amount of Raloxifene is about 10 to about 80 μM.

A fifth embodiment is a method wherein the amount of Raloxifene is about 40 to about 80 μM.

A sixth embodiment is a method wherein the amount of Raloxifene is about 40 μM

A seventh embodiment is a method wherein the YTHDF2 repressor compound is Imatinib.

An eighth embodiment is a method wherein the amount of Imatinib is about 10 to about 40 μM.

A ninth embodiment is a method wherein the amount of Imatinib is about 10 to about 20 μM.

A tenth embodiment is a method wherein the amount of Imatinib is about 10 μM.

An eleventh embodiment is a method wherein the hematopoietic stem cells are in contact with the YTHDF2 repressor compound for about 12 to about 24 hours.

A twelfth embodiment is a method wherein the hematopoietic stem cells are in contact with the YTHDF2 repressor compound for about 16 to about 20 hours.

A thirteenth embodiment is a method wherein the hematopoietic stem cells are in contact with the YTHDF2 repressor compound for about 20 hours.

A fourteenth embodiment is a method of preparing hematopoietic stem cells for homing during stem cell transplantation, comprising contacting hematopoietic stem cells with an FTO expression activator compound for about 8 to about 48 hours; and removing the FTO expression activator compound from contact with the hematopoietic stem cells prior to transplantation of the hematopoietic stem cells into a subject.

A fifteenth embodiment is a method wherein the hematopoietic stem cells are harvested from embryonic cord blood.

A sixteenth embodiment is a method The method of claim 14 wherein the FTO expression activator compound is Angiotensin II.

A seventeenth embodiment is a method wherein the amount of Angiotensin II is about 2 μM to about 10 μM.

An eighteenth embodiment is a method wherein the amount of Angiotensin II is about 4 μM,

A nineteenth embodiment is a method wherein the hematopoietic stem cells are in contact with the FTO expression activator compound for about 20 to about 24 hours.

A twentieth embodiment is a method wherein the hematopoietic stem cells are in contact with the FTO expression activator compound for about 20 hours.

### BRIEF DESCRIPTION OF THE FIGURES

FIG. 1A. Graphs depicting relative CXCR4 mRNA expression in human CB CD34⁺ cells after YTHDF1, YTHDF2 and YTHDF3 downregulation or FTO and ALKBH5 overexpression (n=5). Data shown as mean±s.e.m. *P<0.05; **P<0.01; ***P<0.001 by one-way ANOVA.

FIG. 1B. Graphs showing quantification of mean fluorescence intensity (MFI) of surface CXCR4 expression on human CB CD34+ cells (n=5). Data shown as mean±s.e.m. *P<0.05; **P<0.01; ***P<0.001 by one-way ANOVA.

FIG. 1C. Graphs showing relative YTHDF2 binding level of CXCR4, verified by YTHDF2 RIP combined with RT-qPCR (n=3). Data shown as mean±s.e.m. *P<0.05; **P<0.01; ***P<0.001 by one-way ANOVA.

FIG. 1D. Graph illustrating impact of m6A enrichment on CXCR4, verified by m6A IP combined with RT-qPCR (n=3). Data shown as mean±s.e.m. *P<0.05; **P<0.01; ***P<0.001 by one-way ANOVA.

FIG. 1E. Graph illustrating the representative mRNA profile of CXCR4 after actinomycin D treatment in control and sh-YTHDF2 transduced human CB CD34⁺ cells. Data shown as mean±s.e.m. *P<0.05; **P<0.01; ***P<0.001 by one-way ANOVA.

FIG. 1F. Graph showing the relative YTHDF2 binding level of CXCR4 in FTO overexpressing human CB CD34⁺ cells, verified by YTHDF2 RIP combined with RT-qPCR (n=3). Data shown as mean±s.e.m. *P<0.05; **P<0.01; ***P<0.001 by one-way ANOVA.

FIG. 1G. Graph depicting m6A enrichment on relative CXCR4 levels, verified by m6A IP combined with RT-qPCR (n=3). Data shown as mean±s.e.m. *P<0.05; **P<0.01; ***P<0.001 by one-way ANOVA.

FIG. 1H. Graph illustrating the representative mRNA profile of CXCR4 after actinomycin D treatment in control and FTO overexpressing human CB CD34+ cells. Data shown as mean±s.e.m. *P<0.05; **P<0.01; ***P<0.001 by one-way ANOVA.

FIG. 2A. Graph depicting relative YTHDF2 expression level in human CB CD34⁺ cells after YTHDF2 knockdown (n=5). Data shown as mean±s.e.m. *P<0.05; **P<0.01; ***P<0.001 by one-way ANOVA.

FIG. 2B. Histogram showing intracellular flow validation of YTHDF2 knockdown in human CB CD34⁺ cells.

FIG. 2C. Graph depicting relative CXCR4 protein level in human CB CD34⁺ cells after YTHDF2 knockdown (n=5). Data shown as mean±s.e.m. *P<0.05; **P<0.01; ***P<0.001 by one-way ANOVA.

FIG. 2D. Graph showing YTHDF2 binding level of CXCR4 in Control or si-YTHDF2 transfected human CB CD34⁺ cells, verified by YTHDF2 RIP combined with RT-qPCR (n=3). Data shown as mean±s.e.m. *P<0.05; **P<0.01; ***P<0.001 by one-way ANOVA.

FIG. 2E. Graph depicting m6A enrichment on CXCR4, verified by m6A IP combined with RT-qPCR (n=3). Data shown as mean±s.e.m. *P<0.05; **P<0.01; ***P<0.001 by one-way ANOVA.

FIG. 2F. Histogram of surface CXCR4 protein level of Control or si-YTHDF2 transfected human CB CD34⁺ cells. Representative histogram from five independent experiments is shown.

FIG. 2G. Graph depicting quantification of mean fluorescence intensity (MFI) of surface CXCR4 protein level of Control or si-YTHDF2 transfected human CB CD34⁺ cells. Data shown as mean±s.e.m. *P<0.05; **P<0.01; ***P<0.001 by one-way ANOVA.

FIG. 2H. Graph depicting quantification of mean fluorescence intensity (MFI) of surface CXCR4 of Control or si-YTHDF2 transfected human HSCs (CD34⁺ CD38⁻ CD45RA⁻ CD90⁺ CD49f⁺). Data shown as mean±s.e.m. *P<0.05; **P<0.01; ***P<0.001 by one-way ANOVA.

FIG. 2I. Graph illustrating chemotaxis of human CB CD34+ cells toward human recombinant SDF-1, as quantified by flow cytometry. Data pooled from three independent experiments are shown (each dot represents an independent chemotaxis). Data shown as mean±s.e.m. *P<0.05; **P<0.01; ***P<0.001 by one-way ANOVA.

FIG. 2J. Graph illustrating migration of human phenotypic HSCs in CB CD34+ cells toward human recombinant SDF-1 (50 ng/mL), as quantified by flow cytometry. The migration percentage of HSCs was calculated by analyzing the HSC (CD34⁺ CD38⁻ CD45RA⁻ CD90⁺ CD49f⁺) frequency using flow cytometry. Data pooled from five independent experiments are shown (each dot represents an independent chemotaxis). Data shown as mean±s.e.m. *P<0.05; **P<0.01; ***P<0.001 by one-way ANOVA.

FIG. 2K. Graph illustrating the percentage of human CD45⁺ cells in the BM of NSG mice 24 h after transplantation with 500,000 CB CD34+ cells that had been transfected with Control or si-YTHDF2. CD34⁺ cells from three CB samples (CB 1-CB 3) were tested (n=5 mice per group). Data shown as mean±s.e.m. *P<0.05; **P<0.01; ***P<0.001 by one-way ANOVA.

FIG. 2L. Representative flow cytometric analysis of human cells in the BM of NSG mice, 24 hours after transplantation. Left is from a mouse without transplantation (negative control), center and right are from mice transplanted with human CB CD34+ cells transfected with Control or si-YTHDF2. Human engraftment was assessed as the percentage of human CD45⁺ cells.

FIG. 3A. Representative flow cytometric analysis of human engraftment in the BM of NSG mice, 4 months after transplantation. Left is from a mouse without transplantation (negative control), center and right are from mice transplanted with human CB CD34+ cells transfected with Control or si-YTHDF2 Human engraftment was assessed as the percentage of human CD45⁺ cells.

FIG. 3B. Graph depicting the percentage of human CD45+ cells in the PB and BM of NSG mice at the indicated time points after transplantation with 10,000 CB CD34⁺ cells that had been transfected with Control or si-YTHDF2 (n=5 in each group). Data shown as mean±s.e.m. *P<0.05; **P<0.01; ***P<0.001 by one-way ANOVA.

FIG. 3C. Graph depicting the percentage of human CD33⁺ myeloid cell and CD19⁺ B cell in BM was determined 4 month after transplantation. Data shown as mean±s.e.m. *P<0.05; **P<0.01; ***P<0.001 by one-way ANOVA.

FIG. 3D. Poisson statistical analysis. n=30 mice in total. Shapes represent the percentage of negative mice for each dose of cells. Solid lines indicate the best-fit linear model for each data set. Dotted lines represent 95% confidence intervals.

FIG. 3E. Graph illustrating the HSC frequencies (line in the box) and 95% confidence intervals (box) presented as the number of SRCs in 1×10⁶ CD34⁺ cells. Data shown as mean±s.e.m. *P<0.05; **P<0.01; ***P<0.001 by one-way ANOVA.

FIG. 3F. Graph illustrating the human CD45⁺ cell chimerism in the BM of secondary recipient NSG mice at 4 months, which had been transplanted with 5×10⁶ BM cells from primary recipient NSG mice. Data shown as mean±s.e.m. *P<0.05; **P<0.01; ***P<0.001 by one-way ANOVA.

FIG. 4A. Graph depicting relative YTHDF2 expression level in mir-145 transduced human CB CD34⁺ cells (n=3). Data shown as mean±s.e.m. *P<0.05; **P<0.01; ***P<0.001 by one-way ANOVA.

FIG. 4B. Graph depicting quantification of mean fluorescence intensity (MFI) of surface CXCR4 of human CB CD34⁻ cells (n=5). Data shown as mean±s.e.m. *P<0.05; **P<0.01; ***P<0.001 by one-way ANOVA.

FIG. 4C. Graph illustrating mean fluorescence intensity (MFI) of cell surface CXCR4 protein level on human CB CD34⁺ cells after treatment for 20 h with compounds. The concentration of each compound used in this screen was 10 μM unless otherwise stated. They axis represents relative fluorescence units (RFU) calculated by FlowJo.

FIG. 4D. Graph depicting relative YTHDF2 expression level in representative compounds treated human CB CD34⁺ cells (n=5). Data shown as mean±s.e.m. *P<0.05; **P<0.01; ***P<0.001 by one-way ANOVA.

FIG. 4E. Graph depicting relative mir-145 expression level in representative compounds treated human CB CD34⁺ cells (n=5). Data shown as mean±s.e.m. *P<0.05; **P<0.01; ***P<0.001 by one-way ANOVA.

FIG. 4F. Graph depicting the mean fluorescence intensity (MFI) of cell surface CXCR4 protein level on human CB CD34⁺ cells after treatment for 20 h with representative compounds. Data shown as mean±s.e.m. *P<0.05; **P<0.01; ***P<0.001 by one-way ANOVA.

FIGS. 4G-J. Graphs depicting the mean fluorescence intensity (MFI) of cell surface CXCR4 on representative compounds treated human CB CD34⁺ cells combine with Control or sh-mir-145.

FIG. 4K. Graphs illustrating raloxifene (40 μM, 20 h) treated human CB CD34⁺ cells (left) and phenotypic HSC (right) migration toward human recombinant SDF-1, as quantified by flow cytometry. Data pooled from three independent experiments are shown. Data shown as mean±s.e.m. *P<0.05; **P<0.01; ***P<0.001 by one-way ANOVA.

FIG. 4L. Graphs illustrating Imatinib (10 μM, 20 h) treated human CB CD34⁺ cells (left) and phenotypic HSC (right) migration toward human recombinant SDF-1, as quantified by flow cytometry. Data pooled from three independent experiments are shown. Data shown as mean±s.e.m. *P<0.05; **P<0.01; ***P<0.001 by one-way ANOVA.

FIG. 4M. Graph depicting the percentage of human CD45⁺ cells in the BM of NSG mice 24 h after transplantation with 500,000 CB CD34+ cells that had been treated with DMSO or Raloxifene (40 μM, 20 h) (n=3 in each group). Data shown as mean±s.e.m. *P<0.05; **P<0.01; ***P<0.001 by one-way ANOVA.

FIG. 4N. Graph depicting the percentage of human CD45⁺ cells in the BM of NSG mice 24 h after transplantation with 500,000 CB CD34+ cells that had been treated with DMSO or Imatinib (10 μM, 20 h) (n=3 in each group). Data shown as mean±s.e.m. *P<0.05; **P<0.01; ***P<0.001 by one-way ANOVA.

FIG. 4O. Graph depicting the colony output of DMSO or Raloxifene (40 μM, 20 h) treated CB CD34⁺ cells. (n=3). Data shown as mean±s.e.m. *P<0.05; **P<0.01; ***P<0.001 by one-way ANOVA.

FIG. 4P. Graph depicting the colony output of DMSO or Imatinib (10 μM, 20 h) treated CB CD34⁺ cells. (n=3). Data shown as mean±s.e.m. *P<0.05; **P<0.01; ***P<0.001 by one-way ANOVA.

FIG. 5A. Representative flow cytometric analysis of human engraftment in the BM of NSG mice, 4 months after transplantation. Left is from a mouse without transplantation (negative control), center and right are from mice transplanted with human CB CD34⁺ cells treated with DMSO control, Imatinb (10 μM) or Raloxifene (40 μM) for 20 h. Human engraftment was assessed as the percentage of human CD45⁺ cells.

FIG. 5B. Graph depicting the percentage of human CD45⁺ cells, B-cell (CD19⁺), and myeloid cell (CD33⁺) chimerism in the BM of NSG mice after transplantation with 10,000 CB CD34+ cells that had been treated with DMSO or Raloxifene. (n=5). Data shown as mean±s.e.m. *P<0.05; **P<0.01; ***P<0.001 by one-way ANOVA.

FIGS. 5C and 5D. Graphs illustrating the frequency of human SRCs in CB CD34+ cells treated with DMSO or Raloxifene.

FIG. 5E. Graph depicting the percentage of human CD45⁺ cells, B-cell (CD19⁺), and myeloid cell (CD33⁺) chimerism in the BM of NSG mice after transplantation with 10,000 CB CD34+ cells that had been treated with DMSO or Imatinib. (n=5). Data shown as mean±s.e.m. *P<0.05; **P<0.01; ***P<0.001 by one-way ANOVA.

FIGS. 5F and 5G. Graphs illustrating the frequency of human SRCs in CB CD34+ cells treated with DMSO or Imatinib.

FIGS. 5H and 5I. Graphs illustrating Human CD45⁺ cell chimerism in the BM of secondary recipient NSG mice at 4 months, which had been transplanted with 5×10⁶ BM cells from primary recipient NSG mice (n=5 in each group). Data shown as mean±s.e.m. *P<0.05; **P<0.01; ***P<0.001 by one-way ANOVA.

FIG. 6A. Graph illustrating relative FTO expression level in vehicle (PBS) or Angiotensin II (AGII, 4 μM) treated human CB CD34⁺ cells (n=5). Data shown as mean±s.e.m. *P<0.05; **P<0.01; ***P<0.001 by one-way ANOVA.

FIG. 6B. Graph illustrating YTHDF2 binding level of CXCR4 in vehicle or AGII treated human CB CD34⁺ cells, verified by YTHDF2 RIP combined with RT-qPCR (n=5). Data shown as mean±s.e.m. *P<0.05; **P<0.01; ***P<0.001 by one-way ANOVA.

FIG. 6C. Graph illustrating m6A enrichment on CXCR4 in vehicle or AGII treated human CB CD34⁺ cells, verified by m6A IP combined with RT-qPCR (n=5). Data shown as mean±s.e.m. *P<0.05; **P<0.01; ***P<0.001 by one-way ANOVA.

FIG. 6D. Graph depicting the quantification of mean fluorescence intensity (MFI) of surface CXCR4 protein level of human CB CD34⁺ cells treated with vehicle or AGII. Data pooled from three independent experiments are shown (n=3). Data shown as mean±s.e.m. *P<0.05; **P<0.01; ***P<0.001 by one-way ANOVA.

FIG. 6E. Histogram of surface CXCR4 expression of human CB CD34⁺ cells treated with vehicle or AGII. Representative histogram from three independent experiments is shown.

FIG. 6F. Graph depicting the quantification of mean fluorescence intensity (MFI) of surface CXCR4 of human CB CD34⁺ cells treated with representative compounds. Data pooled from three independent experiments are shown (n=3).

FIG. 6G. Graph illustrating the relative FTO expression level in representative group of human CB CD34⁺ cells (n=5). Data shown as mean±s.e.m. *P<0.05; **P<0.01; ***P<0.001 by one-way ANOVA.

FIG. 6H. Histogram of surface CXCR4 expression in representative group of human CB CD34⁺ cells.

FIG. 6I. Graph depicting the quantification of mean fluorescence intensity (MFI) of surface CXCR4 of human CB CD34⁺ cells treated with representative compounds and shRNA. Data pooled from five independent experiments are shown.

FIG. 6J. Graph illustrating the colony output of vehicle or AGII treated CB CD34⁺ cells. (n=3).

FIG. 6K. Graph illustrating chemotaxis of human CB CD34⁺ cells (left) and phenotypic HSC (right) migration toward human SDF-1, as quantified by flow cytometry. Data pooled from six independent experiments are shown (each dot represents an independent chemotaxis). Data shown as mean±s.e.m. *P<0.05; **P<0.01; ***P<0.001 by one-way ANOVA.

FIG. 6L. Graph depicting the percentage of human CD45⁺ cells in the BM of NSG mice 24 h after transplantation with 500,000 CB CD34⁺ cells that had been treated with vehicle or AGII (n=3 mice per group). Data shown as mean±s.e.m. *P<0.05; **P<0.01; ***P<0.001 by one-way ANOVA.

FIG. 6M. Graph illustrating the percentage of human CD45⁺ cells in the PB and BM of NSG mice at the indicated time points after transplantation with 10,000 CB CD34⁺ cells that had been treated with vehicle or AGII (n=5). Data shown as mean±s.e.m. *P<0.05; **P<0.01; ***P<0.001 by one-way ANOVA.

FIG. 6N. Graph illustrating the percentage of human CD33⁺ myeloid cells and human CD19⁺ cells in the BM of NSG mice 4 months after transplantation with 10,000 CB CD34⁺ cells that had been treated with vehicle or AGII (n=5 mice per group). Data shown as mean±s.e.m. *P<0.05; **P<0.01; ***P<0.001 by one-way ANOVA.

FIGS. 6O and 6P. Graphs depicting the frequency of human SRCs in CB CD34⁺ cells treated with vehicle or AGII, as determined by transplantations of graded doses of treated cells into NSG mice and determination of human CD45⁺ cell chimerism 3 months after transplantation (n=30 mice in total).

FIG. 6Q. Graph illustrating the Human CD45⁺ cell chimerism in the BM of secondary recipient NSG mice at 4 months, which had been transplanted with 5×10⁶ BM cells from primary recipient NSG mice (n=5 per group). Data shown as mean±s.e.m. *P<0.05; **P<0.01; ***P<0.001 by one-way ANOVA.

FIG. 7A. Graph depicting quantification of mean fluorescence intensity (MFI) of surface CXCR4 of human CB CD34+ cells treated with Raloxifene for different time points. Representative data from two independent experiments are shown (n=5 cultures per group, one-way ANOV). *P<0.01; ***P<0.001 when compared with DMSO control.

FIG. 7B. Graph depicting quantification of mean fluorescence intensity (MFI) of surface CXCR4 of human CB CD34+ cells treated with DMSO, different doses of Raloxifene. Representative data from two independent experiments are shown (n=5 cultures per group, one-way ANOV). *P<0.01; ***P<0.001 when compared with DMSO control.

FIG. 7C. Graph depicting quantification of mean fluorescence intensity (MFI) of surface CXCR4 of human CB CD34+ cells treated with Imatinib for different time points. Representative data from two independent experiments are shown (n=5 cultures per group, one-way ANOV). *P<0.01; ***P<0.001 when compared with DMSO control.

FIG. 7D. Graph depicting quantification of mean fluorescence intensity (MFI) of surface CXCR4 of human CB CD34+ cells treated with DMSO, different doses of Imatinib. Representative data from two independent experiments are shown (n=5 cultures per group, one-way ANOV). *P<0.01; ***P<0.001 when compared with DMSO control.

### DETAILED DESCRIPTION

For purposes of promoting an understanding of the principles of the novel technology, reference will be made to embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

Homing efficiency of HSCs is the key factor for successful HCT based clinical therapy. CXCR4 is one of the most important mediators in regulating the homing of HSCs. Two important regulators of human CB CD34⁺ cell homing and engraftment are YTHDF2 (YTH (N6-Methyladenosine RNA Binding Protein 2)) and FTO (fat mass- and obesity-associated gene). Repression of YTHDF2 or activation of FTO in human CB CD34⁺ cells lead to upregulation of CXCR4 expression, thereby facilitating HSC migration, homing and engraftment.

N⁶-methyladenosine (m⁶A) is a type of prevalent modification in cellular mRNA and this reversible modification is associated with RNA maturation, translation, localization and decay (Li, et al., Annu Rev Genomics Hum Genet 2014; 15:127-150; Roundtree, et al., Cell 2017 Jun. 15; 169(7):1187-1200). While several reports described the function of m⁶A modification in the development of HSCs, the role of this modification on HSC homing has never been elucidated (Mapperley, et al., J Exp med 2021 Mar. 1; 218(3); Peritz, et al., Nat Protoc 2006; 1(2):577-580). We show herein the impact m⁶A modification on CXCR4 in HSCs and the function of this modification in HSC homing and engraftment. Our work shows that inhibition of CXCR4 decay by downregulation of YTHDF2, a m⁶A reader or upregulation of FTO, a m6A eraser, promotes HSC homing and engraftment. This study elucidates a novel function of m⁶A modification in HSC homing and engraftment and provides a new regulation strategy for future clinical applications.

The invention described herein includes methods of enhancing homing and engraftment of HSC in transplantation. The method include transiently exposing the HSCs to compounds to repress YTHDF2 expression or to compounds to activate FTO expression, washing off the compound from the HSCs after the period of exposure and using the treated cells in HCT. The invention results in improved reconstitution of human blood system.

As unless explicitly stated otherwise or clearly implied otherwise the term ‘about’ when used in conjunction with a numerical value means plus or minus 10 percent (%). For example, the term about 1.0 encompasses all values between and including 0.9 to 1.1.

Unless stated otherwise, the term “therapeutically effective amount” refers to an amount of a pharmaceutically active compound that when administered to a human being or an animal patient or to a cell or collection of cells either alone or in combination with other pharmaceutically active ingredients or other components of medicaments that have a desirable effect on the physiological condition of a patient or the cell or collection of cells.

As used herein Hematopoietic stem cells (HSCs) are cells typically positive for CD34 (CD34+ cells). While CD34+ is the most common marker used to identify HSCs, other markers known to those of skill in the art to identify less differentiated forms of progenitor cells and/or stem cells are also encompassed within the scope of the invention. HSCs may be harvested from mobilized peripheral blood cells from the bone marrow of a donor or of a patient or from umbilical cord blood. While HSCs harvested from any of these sources may be utilized in the methods of the invention, HSCs harvested from umbilical cord blood are preferred. As used herein, the term harvested refers to any process of isolating or separating the desired HSCs from the other components of the blood source.

As used herein the terms homing and migration refer to the process by which HSC return from the peripheral blood to the bone marrow niche.

As used herein, the term engraftment refers to the reestablishment of the HSC within the bone marrow niche.

As used herein, the term YTHDF2 repressor compound includes compounds that can transiently induce and maintain downregulation of YTHDF2. Compounds that are capable of downregulating YTHDF2 include those identified as capable of overexpression of the mir-145 gene. Preferably, the YTHDF2 repressor compound is Raloxifene or Imatinib.

The amount of YTHDF2 repressor compound to which the HSCs are exposed in order to induce and maintain downregulation of YTHDF2 may vary depending upon the specific compound utilized. As the compound is being utilized on a plurality of HSCs being prepared for transplantation and not on the subject being transplanted, a person of skill in the art will appreciate that the therapeutically effective amount of the compound will be less. For example, a therapeutically effective amount of Raloxifene for use in the present invention is 10-80 μM, more preferably 40-80 μM, and most preferably 40 μM. A therapeutically effective amount of Imatinib for use in the present invention is 10-40 μM, and more preferably 10-20 μM, and most preferably 10 μM.

The amount of time to which HSCs are exposed to the YTHDF2 repressor compound in order to induce and maintain downregulation of YTHDF2 may also vary depending upon the specific compound utilized. The HSCs may be evaluated periodically during the time of exposure to determine if YTHDF2 expression has been repressed. Exposure times may be within the range of 8 hours to 48 hours, preferably within the range of 12 hours to 24 hours, within the range of 16 hours to 20 hours, and most preferably 20 hours.

As used herein, the term FTO expression activator compound includes compounds capable of transiently increasing expression of the FTO gene. Preferably, the FTO expression activator compound is Angiotensin II (AGII).

The amount of FTO expression activator compound to which the HSCs are exposed in order to activate or upregulate of FTO may vary depending upon the specific compound utilized. As the compound is being utilized on a plurality of HSCs being prepared for transplantation and not on the subject being transplanted, a person of skill in the art will appreciate that the therapeutically effective amount of the compound will be less. For example, a therapeutically effective amount of ANGII for use in the present invention is 2-6 μM, more preferably 4 μM.

The amount of time to which HSCs are exposed to the FTO expression activator compound in order to activate FTO may also vary depending upon the specific compound utilized. The HSCs may be evaluated periodically during the time of exposure to determine if FTO expression has been increased. Exposure time may be within the range of 12 hours to 48 hours, preferably within the range of 20 hours to 24 hours.

### Example 1

### Inhibiting CXCR4 Decay Through Downregulation of YTHDF2 or Upregulation of FTO.

CXCR4 is one of the most important mediators in regulating the homing of HSCs. Therefore, we focused on this gene and hypothesized that the epigenetic regulation on mRNA contributes to the expression of CXCR4 and HSC homing.

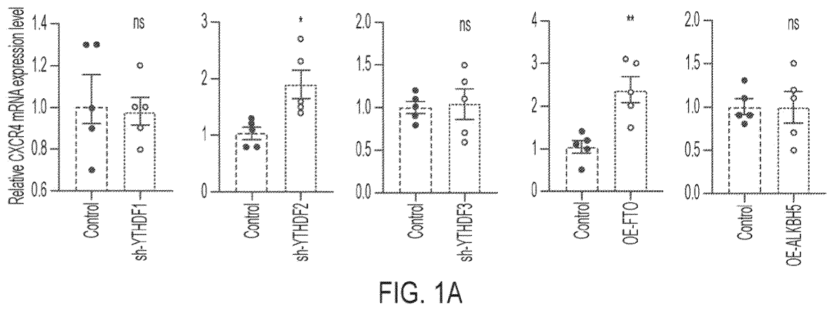

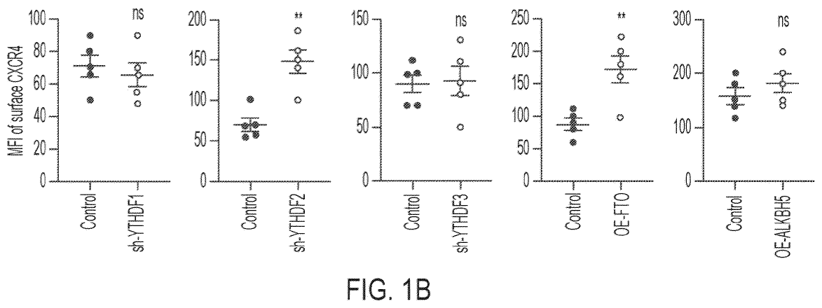

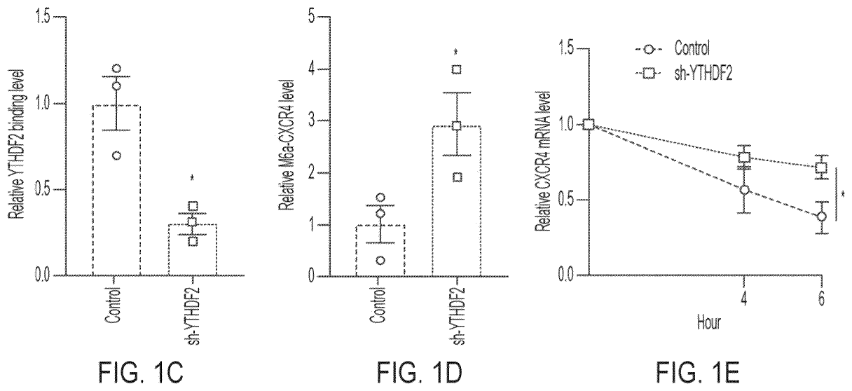

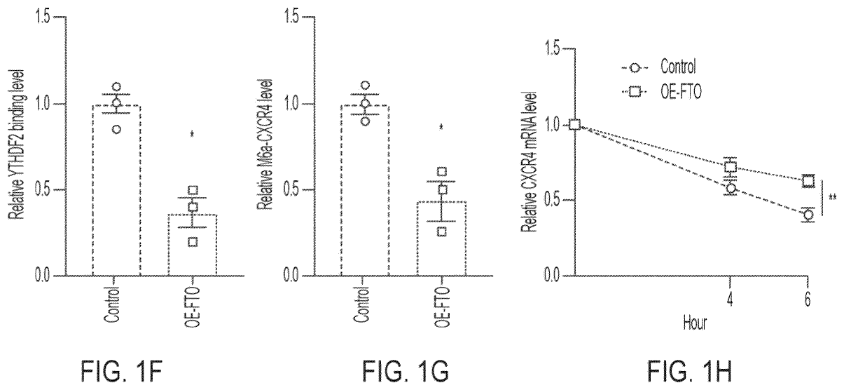

m⁶A modifications in HSCs, N⁶-methyladenosine (m⁶A) is a type of prevalent modification in cellular mRNA and this reversible modification is associated with RNA maturation, translation, localization and decay (Li and Mason, Annu Rev Genomics hum Genet 2014; 15:127-150; Roundtree et al., Cell 2017 Jun. 15, 169(7):1187-1200). m⁶A-seq data has shown that CXCR4 is labelled by m⁶A in human CB CD34⁺ cells (Li, et al., Cell Res 2018; 28(9): 904-917). Based on this, we conducted loss-and-gain of function experiments to explore the regulators that modulate RNA methylation of CXCR4. Five m⁶A regulators were measured in these experiments. As shown previously, YTHDF functions to mediate the decay of m⁶A-mRNAs (Wang et al., Nature 2014 Jan. 2; 505(7481): 117-120; Zaccara and Jaffrey, Cell 2020 Jun. 25; 181(7): 1582-1595 e1518). By using a lentivirus based knockdown strategy, 3 members of the YTH N6-methyladenosine RNA binding protein (YTHDF) family (YTHDF1, YTHDF2 and YTHDF3), were downregulated in CB CD34+ cells. We found that only downregulation of YTHDF2 upregulated CXCR4 expression at RNA level as well at the protein level (FIG. 1A, B). These results suggest that YTHDF2 plays a key role in regulating the decay of CXCR4 mRNA in human CD34⁺ cells.

In contrast, overexpression of two different m⁶A demethylases, FTO and alkB homolog 5 (ALKBH5) by using lentivirus, induced a very different phenotype (Li, et al., Cancer Cell 2017 Jan. 9; 31(1):127-141; Mizuno, TM, Nutrients 2018 Nov. 1; 10(11); Zheng, et al., Mol Cell 2013 Jan. 10; 49(1):18-29). Herein, we show that, overexpression of FTO demethylase but not ALKBH5, promotes CXCR4 expression (FIG. 1A, B). These results suggest that removal of the m⁶A tag from the CXCR4 mRNA attenuates its decay.

In a protein binding assay, we found that when YTHDF2 was knocked down using a shRNA in human CD34⁺ cells, the binding levels of YTHDF2 and CXCR4 were greatly decreased (FIG. 1C) and the relative m⁶A labelled CXCR4 levels were dramatically increased (FIG. 1D).

Actinomycin D is a transcription inhibitor and widely used in mRNA stability assays. Actinomycin D can form a very stable complex with DNA and thus inhibiting the DNA-dependent RNA polymerase activity, allowing the assessment of mRNA stability by measuring their abundance following transcription inhibition (Szeberenyi, J., Biochem Mol Biol Educ 2006 January; 34(1): 50-51. In a mRNA stability assay, after actinomycin D treatment of human CB CD34⁺ cells for 6 hours, the relative CXCR4 mRNA levels were significantly upregulated when YTHDF2 was downregulated by shRNA (FIG. 1E). Our results suggest that YTHDF2 can bind m⁶A labelled CXCR4 in human CB CD34⁺ cells resulting in its decay. Downregulation of YTHDF2 induced lower binding level between YTHDF2 and CXCR4. Eventually, higher expression level of CXCR4 was detected in YTHDF2 downregulated group.

When FTO was overexpressed in human CB CD34⁺ cells, the level of binding between YTHDF2 and CXCR4 was reduced (FIG. 1F), as well as the relative m⁶A labelled CXCR4 level (FIG. 1G). When we treated human CB CD34⁺ cells with actinomycin D for 6 hours, the CXCR4 mRNA levels were significantly increased (FIG. 1H). These results suggest that FTO functions as an “eraser” to remove m⁶A on CXCR4, which inhibits CXCR4 decay by decreasing the binding of YTHDF2 to CXCR4.

### Example 2

Transient Knockdown of YTHDF2 by siRNA Promotes Human HSC Migration and Homing.

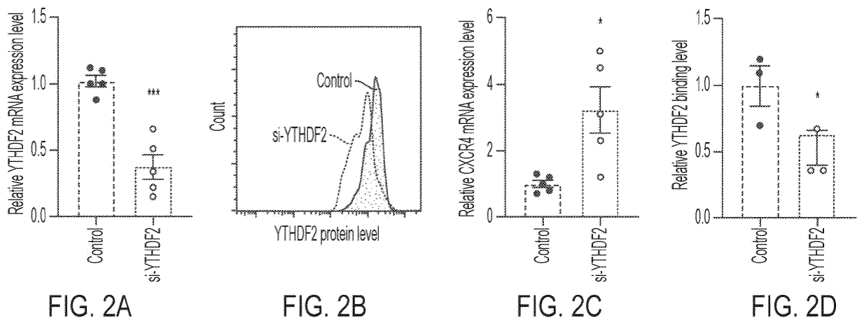

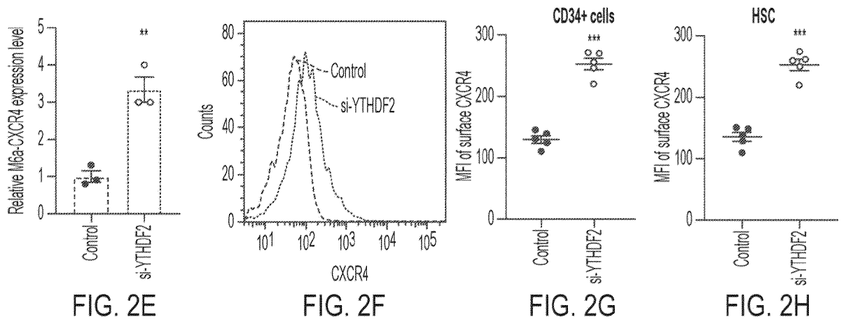

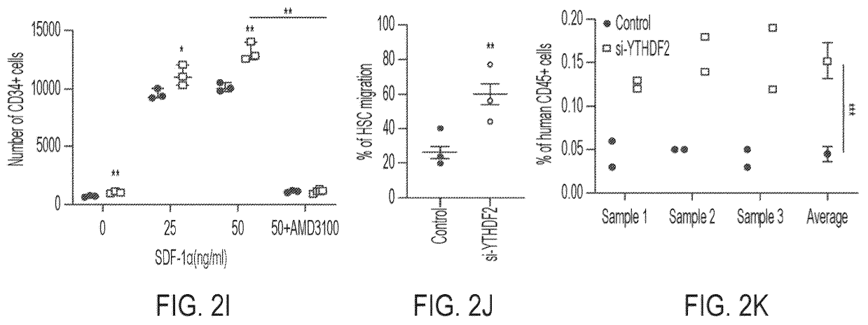

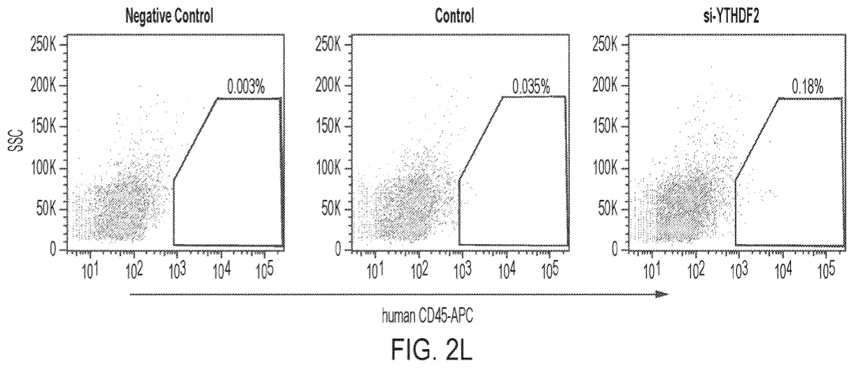

As homing is a short-term process and, in theory, transfection of siRNA into human CB CD34⁺ cells can transiently induce and maintain downregulation of YTHDF2, we transfected human CB CD34⁺ cells with siRNA by electroporation. siRNA efficiently downregulated YTHDF2 expression at the RNA and at the protein level in human CB CD34⁺ cells (FIG. 2A, B). When YTHDF2 was knockdown by siRNA, the RNA level of CXCR4 was significantly upregulated (FIG. 2C). The binding level of YTHDF2 and CXCR4 was significantly decreased (FIG. 2D) and the expression level m⁶A labelled CXCR4 was greatly increased (FIG. 2E). These results suggest that knockdown of YTHDF2 by siRNA is feasible in human CB CD34+ cells. Furthermore, flow cytometry analysis of knockdown cells revealed that CXCR4 protein level was notably upregulated in human CB CD34⁺ cells, as well as in the rigorously defined population of HSCs (CD34⁺ CD38⁻ CD45RA⁻ CD49f⁺ CD90⁺) (FIG. 2F-H). The effect of si-YTHDF2 on HSC chemotaxis was evaluated in an in vitro transwell migration assay. siRNA transfected CB CD34⁺ cells showed significant migration to SDF-1 (FIG. 2I). Enhanced migration to SDF-1 by siRNA transfection was also observed in the HSC population (FIG. 2J). Chemotaxis of human CB CD34⁺ cells to SDF-1 was blocked by CXCR4 antagonist AMD3100, suggesting that the enhanced migration was mediated through CXCR4 (FIG. 2I). To further investigate the role of YTHDF2 knockdown by siRNA on homing, siRNA transfected human CB CD34⁺ cells were transplanted into NSG mice. After 24 hours, the human CD45⁺ cells that homed to the BM were analyzed by flow cytometry. siRNA transfection enhanced human CB CD34⁺ cell homing in NSG mice (FIG. 2K, L). These results suggest that transient knockdown of YTHDF2 by siRNA promotes human HSC migration and homing.

### Example 3

Transient Downregulation of YTHDF2 by siRNA Enhanced Human CB CD34⁺ Cell Engraftment.

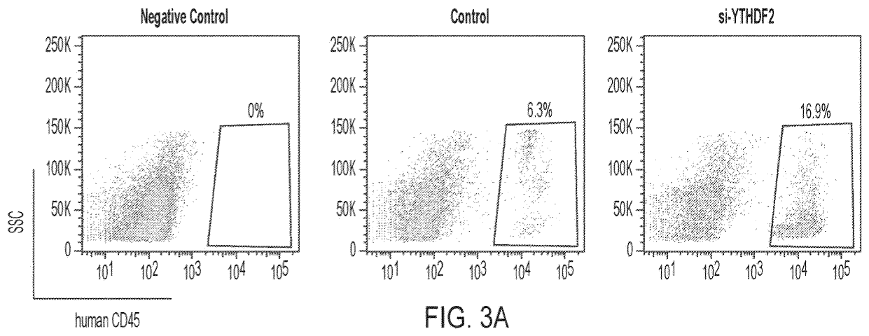

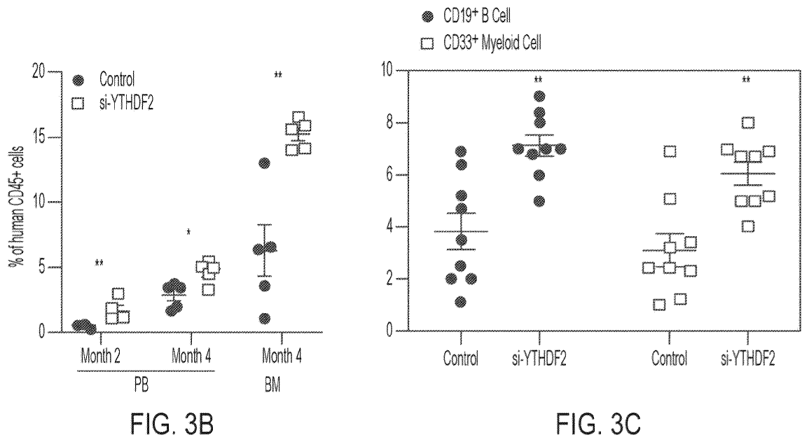

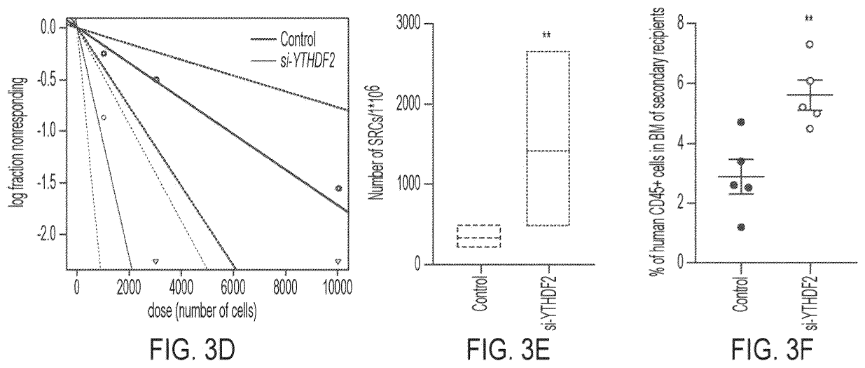

We next evaluated the long-term engraftment of siRNA transfected human CB CD34⁺ cells by limiting dilution assay (LDA). LDA results indicate that siRNA transfected human CB CD34⁺ cells display enhanced engraftment relative to the recipients of control group both in the peripheral blood (PB) at 2 and 4 months, respectively and in the bone marrow (BM) at 4 months post transplantation (FIG. 3A, B). Human myeloid and B-cell chimerism were also significantly increased (FIG. 3C). Human SCID repopulating cells (SRC) were also increase. Poisson distribution analysis revealed a SRC frequency of 1/2439 for the control group and 1/667 for the siRNA transfected group. This reflected respectively 410 and 1478 SRCs in 1×10⁶ cells from control and siRNA transfected group, resulting in a ˜4-fold increase in the number of functionally detectable SRCs compared with the control group (FIG. 3D, E). Transplanting BM cells from the siRNA transfected group into secondary NSG mice, also resulted in the enhanced engraftment (FIG. 3F). These data suggest that the long-term self-renewal capability of human CB HSCs was enhanced by transient siRNA mediated knockdown of YTHDF2.

### Example 4

Identification of Several Compounds that Functionally Downregulate YTHDF2 and Enhance Human CB CD34⁺ Cell Migration and Homing.

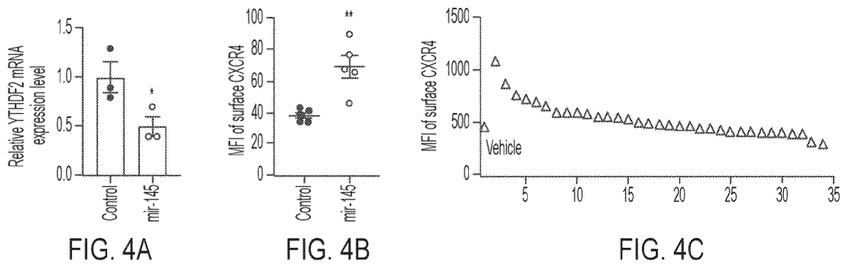

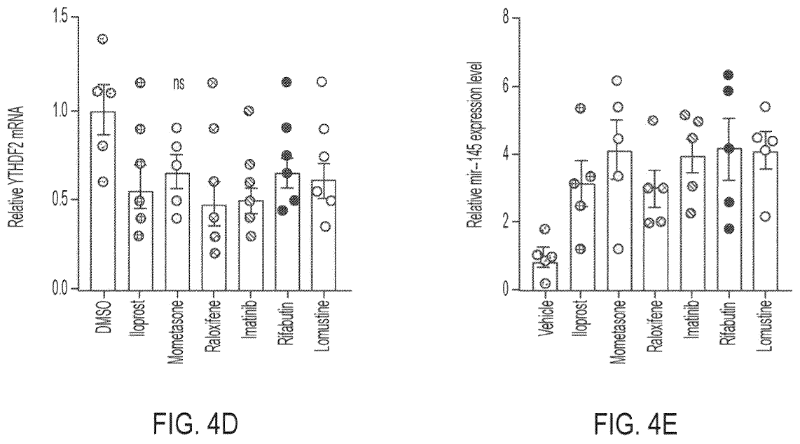

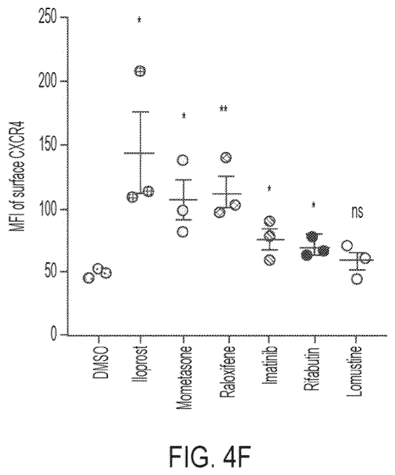

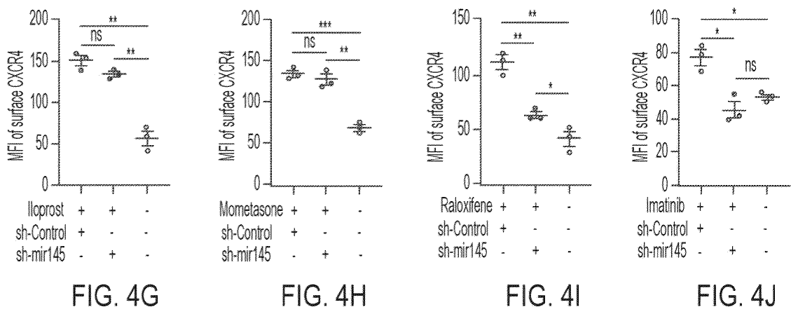

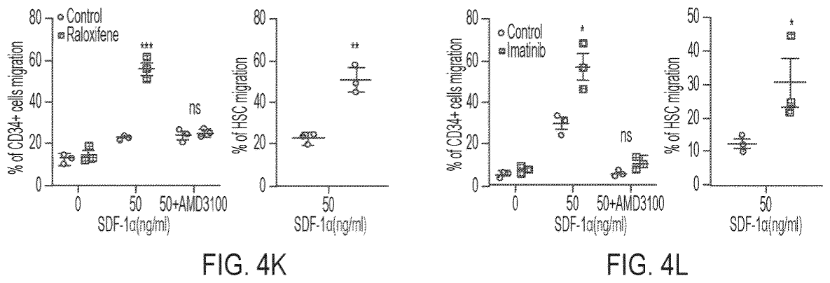

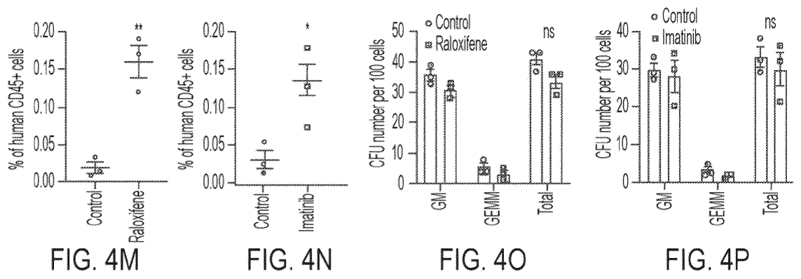

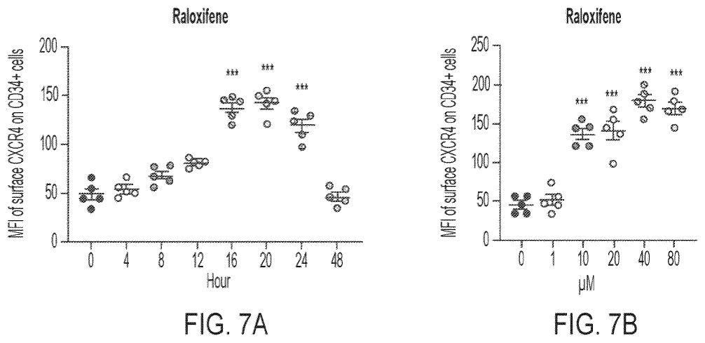

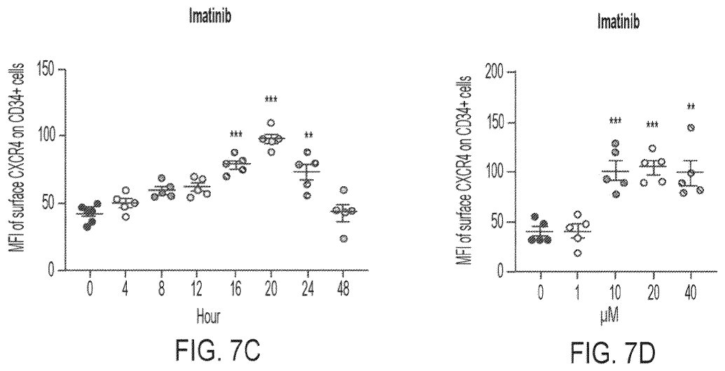

In previous reports, mir-145 was identified as a repressor of YTHDF2 by targeting the 3′-untranslated mRNA region of YTHDF2 (Li et al., J Ovarian Res 2020 Sep. 18; 13(1):111, Yang et al., J Biol Chem 2017 Mar. 3; 292(9):3614-3623). Overexpression of mir-145 in human CB CD34⁺ cells resulted in notable downregulation of YTHDF2 (FIG. 4A). Using flow cytometry, we observed an upregulation of CXCR4 protein level in human HSCs overexpressing miR-145 (FIG. 4B). These results demonstrate that mir-145 can regulate YTHDF2 expression and eventually the expression of CXCR4 in human HSCs. By mining a public database, Psmir (Meng et al., Sci Rep 2016 Jan. 13; 6:19264), we identified 33 candidate compounds that could induce mir-145 expression. We treated human CB CD34⁺ cells with these compounds for 20 hours and assessed the expression of CXCR4, YTHDF2 and mir-145 (FIG. 4C-E). We found Iloprost, Mometasone, Raloxifene and Imatinib strongly upregulated mir-145 and CXCR4 expression and downregulated YTHDF2 expression (FIG. 4D-F). Iloprost is a synthetic analogue of prostacyclin Moreno et al., Eur J Pharmacol 2017 Feb. 5; 796:7-19). It's well known that prostaglandins and their receptors have important function in regulating HSC homing (Hoggatt, et al., Blood 2009 May 28; 113(22):5444-5455). Mometasone, a type of glucocorticoid, is known to boost HSC homing (Guo, et al., Nat Med 2017 April; 23(4): 424-428). When human CB CD34⁺ cells were treated with these compounds and sh-mir-145, we found that inhibition of mir-145 did not affect the function of Iloprost and Mometasone on CXCR4 activation (FIG. 4G, H). In contrast, the impact of Raloxifene and Imatinib on CXCR4 expression in human CB CD34⁺ cells were significantly blocked by inhibition of mir-145 (FIG. 41, J). These results suggest that Raloxifene and Imatinib upregulate CXCR4 expression mainly via activation of mir-145 expression. Iloprost and Mometasone likely regulate CXCR4 expression through more complicated mechanisms as previously reported. The optimal concentration and time of treatment to induce maximal CXCR4 expression by Raloxifene and Imatinib in human CB CD34⁺ cells were evaluated in detail (FIG. 7A-D). We next assessed whether treatment of the CB CD34+ cells with Raloxifene, an FDA approved drug to treat osteoporosis, and Imatinib, an FDA approved drug to treat chronic myelogenous leukemia (CML), can promote chemotaxis of human CB HSCs toward SDF-1. Raloxifene and Imatinib treatment significantly enhanced chemotaxis of human CB CD34⁺ cells toward SDF-1, which could be fully blocked by CXCR4 antagonist AMD3100 (FIG. 4K, L). Enhanced migration to SDF-1 by Raloxifene and Imatinib was also observed in phenotypic HSC population (FIG. 4K, L). We next tested whether Raloxifene and Imatinib treatment of CB CD34+ cells can enhance the homing of these cells in vivo. Human CB CD34⁺ cells were treated with the compounds for 20 hours and then transplanted into NSG mice. We found that both Raloxifene and Imatinib treatment dramatically increased the homing efficiency of human CB CD34⁺ cells (FIG. 4M, N). In colony forming assay, no significant difference was observed in Imatinib and Raloxifene treated cells when compared with the control group, respectively (FIG. 4O, P). Thus, imatinib and Raloxifene treatment promotes human CB CD34⁺ cell migration and homing through downregulation of YTHDF2.

### Example 5

### Raloxifene and Imatinib Treatment Promotes Human CB CD34+ Cell Engraftment.

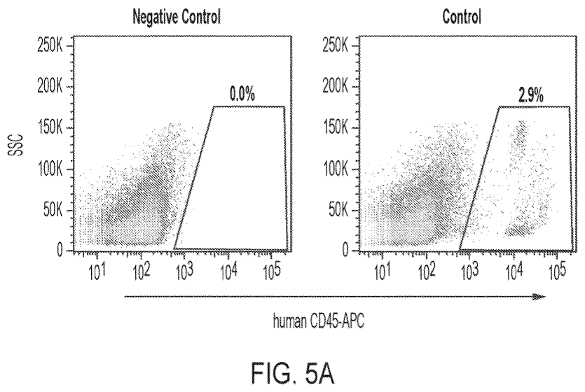

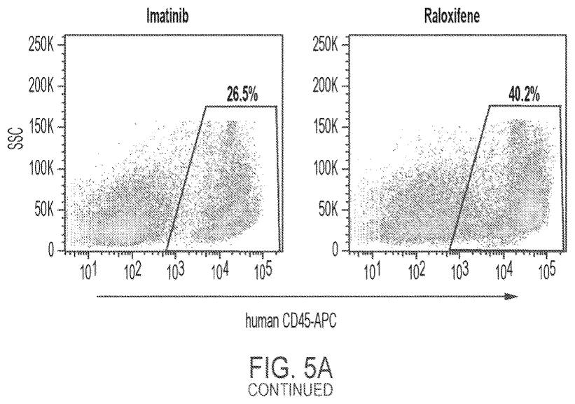

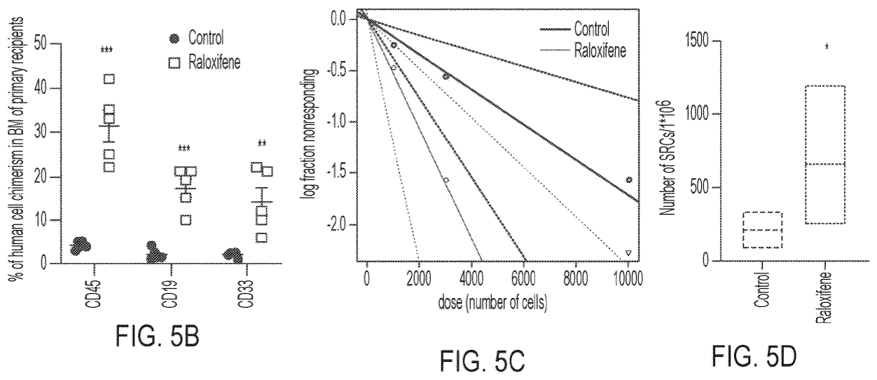

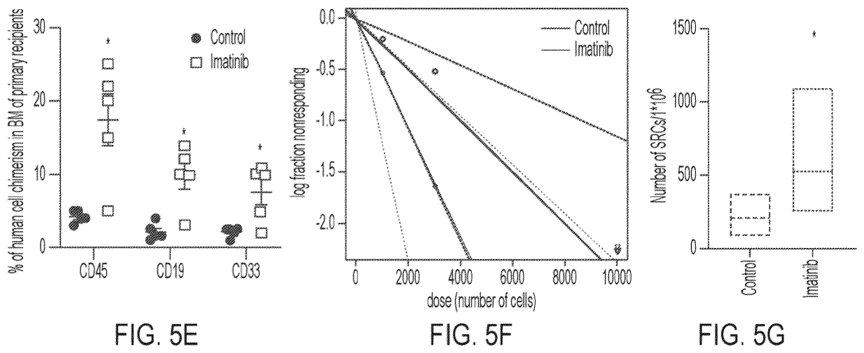

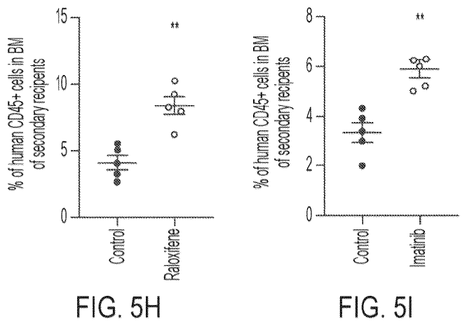

Long-term engraftment of human CB CD34⁺ cells was assessed in recipients transplanted with Imatinib and Raloxifene treated CD34⁺ cells. Imatinib and Raloxifene treated human CB CD34⁺ cells showed significantly elevated engraftment in NSG mice compared with that of the vehicle control-treated group (FIG. 5A). Human myeloid and B-cell chimerism were also increased (FIG. 5B, E). The frequency of SRCs was calculated by LDA. A Poisson distribution analysis revealed an SRC frequency of 1/4761 for vehicle-treated CB CD34⁺ cells and an enhanced SRC frequency of 1/1818 and 1/1406 for Raloxifene and Imatinib treated human CB CD34⁺ cells, respectively. Enhanced engraftment of Imatinib and Raloxifene treated human CB CD34⁻ cells was also apparent in transplanted secondary recipients as compared to vehicle treated cells (FIGS. 5H, I). Thus, treatment of human CB CD34⁺ cells with Raloxifene or Imatinib enhances their long-term engraftment.

### Example 6

### Angiotensin II Promotes Human CD34⁺ Cell Migration and Homing Through Activation of FTO.

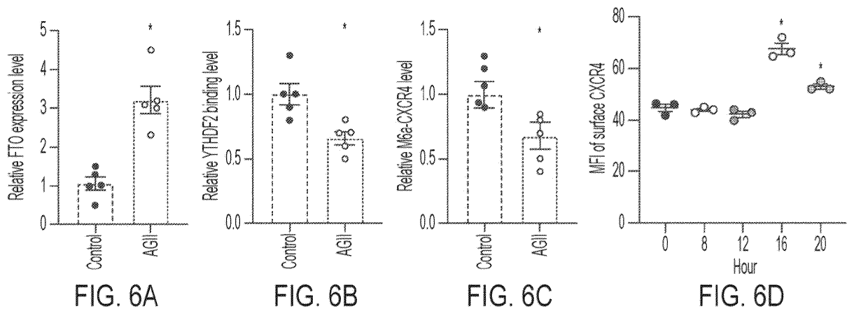

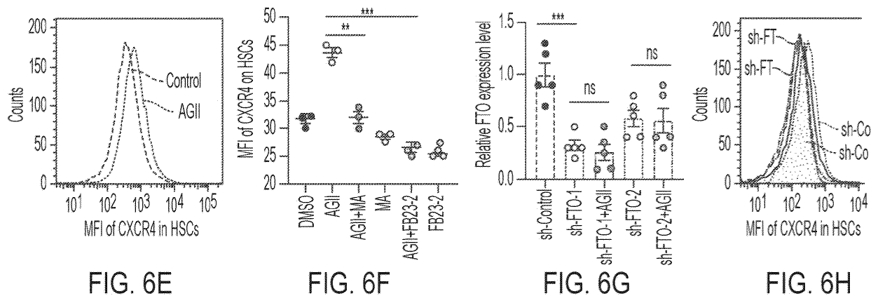

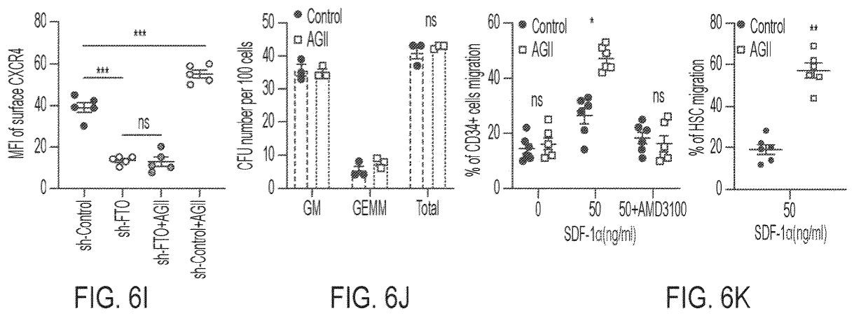

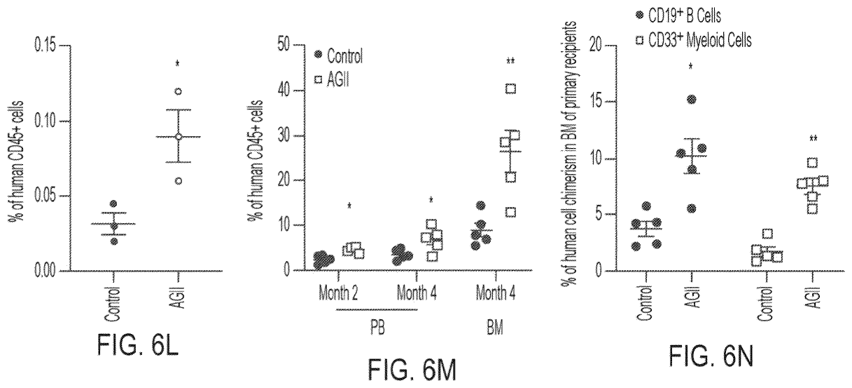

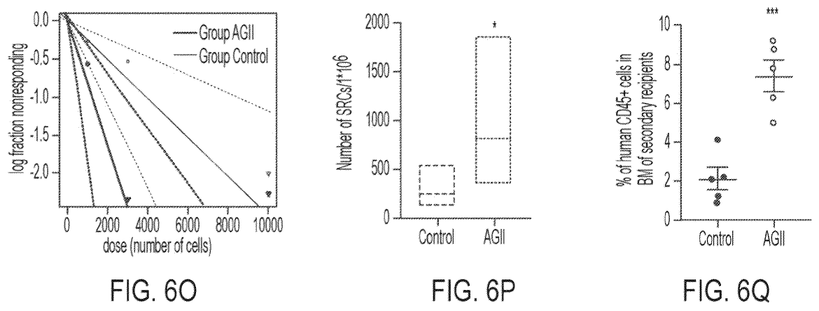

Overexpression of FTO, a m⁶A eraser, upregulates CXCR4 expression in human CB CD34⁺ cells (FIG. 1A, B). Previous studies have shown that Angiotensin II (AGII) can activate FTO expression (Ma et al., Front Cardiovasc Med 2020; 7:592550; Shindo et al., Nat Med 2002 Aug. 8(8):856-863). Angiotensin II is a peptide hormone of the RAAS system being investigated to raise blood pressure in septic or other forms of shock. Therefore, we assessed whether treatment of human CB CD34⁺ cell with AGII can active FTO expression. Using realtime PCR, we found after 20 hours of treatment with AGII, FTO expression was significantly upregulated in human CB CD34⁺ cells (FIG. 6A). The binding between YTHDF2 and CXCR4 was decreased, as well as the relative m6A labeled CXCR4 level in human CB CD34⁺ cells (FIG. 6B, C). These results are consistent with the phenotypes of FTO overexpression in CB CD34+ cells (FIGS. 1F, G). The results of flow cytometry analysis showed that AGII treatment upregulates the protein level of CXCR4 in human HSCs (FIG. 6D, E). To further confirm AGII upregulates CXCR4 expression through activation of FTO in human HSCs, we evaluated the effects of FTO inhibition on AGII treated human HSCs. Inhibition of FTO by Meclofenamic Acid (MA) or FB23-2 repressed the effects of AGII on CXCR4 upregulation (FIG. 6F) (Huang, et al., Cancer Cell 2019 Apr. 15; 35(4):677-691 e610; Huang et al., Nucleic Acids Res 2015 January; 43(1): 373-384). By using shRNA, we found that downregulation of FTO greatly blocked AGII induced upregulation of CXCR4 in human HSCs (FIG. 6G-I). These results demonstrate that AGII upregulates CXCR4 expression in human HSCs through activation of FTO. In colony forming assay, no significant difference was observed between AGII and vehicle treated control group (FIG. 6J). AGII treatment also greatly enhanced human CB CD34⁺ cell migration toward graded doses of SDF-1, as well as chemotaxis to SDF-1 of HSCs (FIG. 6K). To evaluate whether enhanced migration of human CB CD34⁺ cells toward SDF-1 by AGII treatment depends on CXCR4 expression, we used AMD3100. AMD3100 treatment blocked the positive impact of AGII, demonstrating that AGII enhances the migration of human CB CD34⁺ cells via an active SDF-CXCR4 axis (FIG. 6K). In a homing assay, 20 hours of CD34+ cell treatment with AGII dramatically promoted their homing to mouse BM (FIG. 6L). These results show that AGII can activate FTO expression in human CB CD34⁺ cells and eventually promote their migration and homing.

To assess the long-term reconstituting ability of AGII treated human CB CD34⁺ cells, we performed LDA to calculate the frequency of SRCs. AGII treated human CB CD34⁺ cells showed significantly increased engraftment in NSG recipients when compared to the control group, including increased human myeloid and lymphoid cell chimerism (FIGS. 6M, N). The Poisson distribution analysis revealed the SRC frequency of 1/3984 for control human CB CD34⁺ cells and an enhanced SRC frequency of 1/1280 for AGII treated human CB CD34⁺ cells, indicating the presence of 251 and 781 SRCs in control and AGII treated cells, respectively (FIGS. 6O, P). In secondary transplantation, the enhanced engraftment of AGII treated human CB CD34⁺ cells was apparent as compared to control group (FIG. 6Q). Thus, these results suggest that AGII treatment enhances human CB CD34⁺ cells engraftment.

### Materials and Methods Used in the Examples

Human CD34⁺ CB cell collection and culture. Umbilical CB units were supplied by Cleveland Cord Blood Bank and CordUse, Orlando, FL, USA. All studies were approved by the institutional review board of the IUSM. Mononuclear cells were isolated by density gradient centrifugation and CD34⁺ cells were isolated by immunomagnetic selection kit (Miltenyi Biotec, 130-046-702). The purity of human CD34⁺ CB cells was over 90% and the cells were cultured in StemSpanII serum-free medium (STEMCELL Technologies, 09650) supplemented with 100 ng/mL stem cell factor (SCF), 100 ng/mL FMS-like tyrosine kinase 3 ligand (Flt3L) and 100 ng/mL thrombopoietin (TPO). For molecular compound treatment, all reagents were ordered from Cayman Chemical (Ann Arbor, MI, USA). Briefly, molecular compounds were added in the initiation of human CB CD34⁺ cells culture, the time and concentration of each molecular compound for cell treatment was shown in figure legends.

Mice. Immunodeficient 6- to 8-week-old NSG (NOD.Cg-Prkdcscid IL2rgtm1Wj1/SzJ) mice were obtained from the In vivo Therapeutics Core at Indiana University School of Medicine (IUSM). The mice were maintained in the Laboratory Animal Resource Center (LARC) at IUSM. All animal experiments followed protocols approved by the Institutional Animal Care and Use Committee of IUSM.

RNA extraction and real-time PCR. After cells were harvested by FACS, RNA was extracted by a RNeasy Mini Kit following manufacturer's protocol (Qiagen, 74106). Total RNA was reverse-transcribed by use of Superscript III kit (ThermoFisher, 18080093). Quantitative real-time PCR reactions were performed by SYBR Green PCR Master Mix (Thermo Fisher, Florence, KY, USA) and an Agilent Mx3000P QPCR System. Expression of housekeeping gene GAPDH was used as an internal control. Data are shown as relative mRNA level normalized to levels in vehicle control, set to 1. SEQ ID NO: 1-16 are the primer sequences utilized.

siRNA transfection. siRNA oligos were synthesized by Genewiz (South Plainfield, NJ). Non-targeting siRNA was used as the control. The siRNA duplexes sequences used to target YTHDF2 is 5′-TTGGCTATTGGGAACGTCCTT-3′ (SEQ ID NO:17). For electroporation, human CD34⁺ cells were resuspended at 1×10⁶ cell/ml in StemSpanII serum-free medium and thereafter electroporated using the CD34⁺ Cell Nucleofector Kit and Nucleofector II device from Amaxa. Control, si-YTHDF2 or a fluorescently labelled, non-targeting siRNA at a final concentration of 2 μM was added in a volume of 10 μl Nucleofector Solution. Briefly, 200 ul human CD34⁺ cells were pelleted for 5 min at 400×g and resuspended in 10 μl the prepared Nucleofector Solution. Cell suspension was transferred into a cuvette and electroporated with program U-008 for human CD34⁺ cells. After electroporation, 500 μL pre-warmed StemSpanII serum-free medium was added to the cuvette and then the suspension was transferred into a 24-Well plate. In vitro or in vivo assays were performed after 24 hours. Transfection efficiencies were analyzed by calculating the percentage of fluorescent cells regarding the total cell.

RNA immunoprecipitation. RNA immunoprecipitation was performed as previously described¹⁴. Briefly, 0.5-1×10⁶ collected human CD34⁺ cells were lysed in buffer A (20 mM Tris-Cl, pH 8.0; 100 mM NaCl, 1 mM EDTA, 0.5% NP-40, protease and RNase inhibitors) for 20 min at 4° C. After centrifugation for 5 min, 1/10^{th} volume of cell lysate was saved as input. Anti-YTHDF2 (Abcam, ab220163) was prebound to the protein A/G beads and then reacted with the rest of the cell lysate overnight at 4° C. After elution from the beads, the RNA samples were precipitated with ethanol and dissolved in RNase-free water. RNAs were further purified with RNA Clean and Concentrator-5 (Zymo, R1013). The relative CXCR4 enrichment was determined by calculating the Ct values of the RIP sample relative to the input sample. Expression of non-m6a housekeeping gene ACTB was used as an internal control. Data are shown as relative binding level normalized to levels in sh-Control group, set to 1.

M6A-RNA Immunoprecipitation. MeRIP analysis was used as previously described¹⁵. Briefly, the total RNAs were extracted, and then RNA concentration was adjusted to 1 μg/μl. RNA was fragmented into ˜100 nt size and these RNA were immunoprecipitated with the anti-m6A antibody according to the standard protocol of the Magna MeRIP m6A Kit (Merck Millipore, 17-10499). M6A enrichment was determined by qPCR analysis.

Vector construction and virus production. FTO and ALKBH5 cDNA were cloned from CB CD34⁺ cell cDNA library. Briefly, fresh CB CD34⁺ cells were harvested and total RNA extracted. The total RNA was reverse-transcribed by use of Superscript III kit. The cDNA was then ligated into an overexpression plasmid by use of In-Fusion HD Cloning Plus kit (Takara, 638920). For shRNA, microRNA and anti-microRNA vector construction, single-strand oligos were synthesized by Genewiz. After annealing, double-strand oligos were ligated into the shRNA expression plasmid by T4 ligase (NEB, M2622L). Well-constructed vectors were sent to Genewiz (South Plainfield, NJ, USA) for sequencing.

Lentivirus transduction of CB CD34⁺ cells. Lentiviruses were concentrated by 30% percutaneous endoscopic gastrostomy 8000 (PEG8000, Sigma, 1546605). Before transduction, freshly isolated CB CD34+ cells were cultured in stemspanII medium with SCF, Flt3L and TPO for 6 hours, 20 μm CsH was added to the medium and cells were cultured for 16 hours. Lentivirus was added in the same medium at a MOI of 50-200 and incubated for 8 hours, a step which was repeated 3 times. GFP positive cells were identified as lentivirus transduced cells.

Flow cytometry. Fresh or cultured CB cells were sorted for different phenotypes by using the following antibodies. Antibodies were used to detect CD34 (BV786, BD Biosciences, 744741), CD34 (FITC, BD Biosciences, 340668), CD45RA (Taxe-red, BD Biosciences, 562298), CD90 (BV421, BD Biosciences, 562556), CD49f (cy5.5, BD Biosciences, 562495), CD38 (PE, BD Biosciences, 560981). CD3 (BV421, BD Biosciences, 562427), CD33 (PE, BD Biosciences, 555450), CD19 (PE, BD Biosciences, 555413), CXCR4 (APC, BD Biosciences, 555976) and CD45 (APC, BD Biosciences, 555485). Gating strategy of phenotypic HSCs were shown in supplementary FIG. 2B.

Chemotaxis assay. Chemotaxis assays were performed in Costar 24-well trans-well plates with 6.5 mm diameter inserts with 5.0 μm pores (Corning, NY, USA). Briefly, 650 μL of IMDM medium (37° C.) that contained 0.5% bovine serum albumin (Sigma-Aldrich) and SDF-1 (0, 50 ng/mL) was added to the bottom well. Cells were suspended at 1×10⁵ cells/100 μL in IMDM medium and loaded to the upper chamber of the trans-well. Trans-well plates were placed in a 37° C. incubator with 95% humidity and 5% CO₂ for 4 hours. Percent migration was measured using flow cytometry with number of cells in the bottom chamber divided by number of cells placed in the upper chamber. To calculate the percent migration of CB HSCs, phenotypic HSCs was determined by surface staining and flow cytometry analysis. Human CB HSC chemotaxis was calculated as number of HSCs in the bottom chamber divided by number of HSCs loaded in the upper chamber. For AMD3100 administration, cells were treated with 5 μg/mL AMD3100 (239820, Sigma-Aldrich) for 30 min right before the chemotaxis assay.

Homing assay. Homing of human CB CD34⁺ cells was evaluated in NSG mice. Molecular compounds treated or siRNA transfected human CB CD34+ cells (500,000 for each mouse) were intravenously injected into sub-lethally irradiated (350 cGy) NSG mice. After 24 h, these recipient mice were sacrificed, BM cells from each mouse were collected. Cells were stained with anti-human CD45 antibody, then resuspended in 1% formaldehyde buffer. Flow cytometry analysis was performed to determine the percentage of human CD45⁺ cells. BM cells from non-transplanted NSG mice served as negative controls.

Colony-forming unit (CFU) assay. GFP⁺, CD34⁻ cells harvested after cell sorting by FACS were plated in semi-solid methylcellulose culture medium in presence of 30% Fetal bovine serum (FBS) (GE Healthcare, HyClone, SH30071.03), 2 mM 1-glutamine (Lonza, 17-605E), 100 μM β-mercaptoethanol (Sigma, M6250), 1 U/mL erythropoietin (EPO) (R&D Systems, 287-TC-500), 50 ng/mL SCF (R&D Systems, 7466-SC-010/CF), 10 ng/mL IL-3 (R&D Systems, 203-IL-050/CF) and 10 ng/mL GM-CSF (R&D Systems, 7954-GM-010) and were cultured at lower (5%) 02 and at 5% CO₂ in a humidified incubator. Number of CFU-GM- and CFU-GEMM-colonies were scored with an inverted microscope 14 days after culture in semi-solid medium. This culture medium allows detection of CFU-GM- and CFU-GEMM-colonies, but not BFU-E colonies.

Limiting dilution analysis (LDA). Frequency of human SCID repopulating cells (SRCs) was determined by LDA as reported before. Increasing doses of GFP overexpressing CD34⁺ cells (2500, 5000 or 10000 cells) were intravenously injected into sublethally irradiated NSG recipient mice (350 cGy; 137Cs source, single dose). Four months after transplantation, the percentage of GFP⁺ human CD45⁺ cell chimerism was analyzed by immunostaining and flow cytometry. For long-term engraftment assays that assess the self-renewal capacity of HSC, 3×10⁶ BM cells from primary recipients of the 10000-cell group were intravenously transplanted into secondary sub-lethally irradiated NSG recipient mice.

mRNA stability assay. Each sample was harvested at 4 and 6 hours after treatment with actinomycin D (2 μM). Total RNA was isolated with the RNeasy plus mini kit (QIAGEN). The HPRT1 housekeeping gene was used as a loading control.

Statistical analysis. Statistical analysis was performed by use of Microsoft Excel and GraphPad Prism (GraphPad Software, San Diego, CA, USA). Data are shown as mean±s.e.m. (as indicated in the figure legends). One-way ANOVA was used to compare differences in means between more than two groups, as indicated

SEQUENCE LISTING

| | | SEQ |
|---|---|---|
| | | ID |
| Description | Sequence | NO |
| sh-YTHDF1-F | CACCCCCGAAAGAGTTTGAGTGGAAC | 1 |
| primer | TCGAGTTCCACTCAAACTCTTTCGGG | |
| sh-YTHDF1-R | AAAACCCGAAAGAGTTTGAGTGGAAC | 2 |
| primer | TCGAGTTCCACTCAAACTCTTTCGGG | |
| sh-YTHDF2-F | CACCGCAGACTTGCAGTTTAAGTATC | 3 |
| primer | TCGAGATACTTAAACTGCAAGTCTGC | |
| sh-YTHDF2-R | AAAAGCAGACTTGCAGTTTAAGTATC | 4 |
| primer | TCGAGATACTTAAACTGCAAGTCTGC | |
| sh-YTHDF3-F | CACCGCAAGGAAATAAAGTTTCAGTC | 5 |
| primer | TCGAGACTGAAACTTTATTTCCTTGC | |
| sh-YTHDF3-R | AAAAGCAAGGAAATAAAGTTTCAGTC | 6 |
| primer | TCGAGACTGAAACTTTATTTCCTTGC | |
| sh-FTO-F1 | CACCTCACGAATTGCCCGAACATTAC | 7 |
| primer | TCGAGTAATGTTCGGGCAATTCGTGA | |
| sh-FTO-R1 | AAAATCACGAATTGCCCGAACATTAC | 8 |
| primer | TCGAGTAATGTTCGGGCAATTCGTGA | |
| sh-FTO-F2 | CACCCCCATTAGGTGCCCATATTTAC | 9 |
| primer | TCGAGTAAATATGGGCACCTAATGGG | |
| sh-FTO-R2 | AAAACCCATTAGGTGCCCATATTTACT | 10 |
| primer | CGAGTAAATATGGGCACCTAATGGG | |
| GAPDH-F | TGTGGGCATCAATGGATTTGG | 11 |
| primer | | |
| GAPDH-R | ACACCATGTATTCCGGGTCAAT | 12 |
| Primer | | |
| ACTB-F | ACCGGGCATAGTGGTTGGA | 13 |
| primer | | |
| ACTB-R | ATGGTACACGGTTCTCAACATC | 14 |
| primer | | |
| Mir-145-F | GTCCAGTTTTCCCAGG | 15 |
| primer | | |
| Mir-145-R | GCGAGCACAGAATTAA | 16 |
| primer | | |
| siRNA | TTGGCTATTGGGAACGTCCTT | 17 |
| duplex | | |
| sequences | | |
| targeting | | |
| YTHDF2 | | |

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. All patents, patent applications, and references to texts, scientific treatises, publications, and the like referenced in this application are incorporated herein by reference in their entirety.

I claim:

1. A method of preparing hematopoietic stem cells for homing during stem cell transplantation, comprising
a. contacting hematopoietic stem cells with a YTHDF2 repressor compound for about 8 to about 48 hours; and
b. removing the YTHDF2 repressor compound from contact with the hematopoietic stem cells prior to transplantation of the hematopoietic stem cells into a subject.

2. The method of claim 1 wherein the hematopoietic stem cells are harvested from embryonic cord blood.

3. The method of claim 1 wherein the YTHDF2 repressor compound is Raloxifene.

4. The method of claim 3 wherein the amount of Raloxifene is about 10 to about 80 μM.

5. The method of claim 4 wherein the amount of Raloxifene is about 40 to about 80 μM.

6. The method of claim 5 wherein the amount of Raloxifene is about 40 μM

7. The method of claim 1 wherein the YTHDF2 repressor compound is Imatinib.

8. The method of claim 7 wherein the amount of Imatinib is about 10 to about 40 μM.

9. The method of claim 8 wherein the amount of Imatinib is about 10 to about 20 μM.

10. The method of claim 9 wherein the amount of Imatinib is about 10 μM.

11. The method of claim 1 wherein the hematopoietic stem cells are in contact with the YTHDF2 repressor compound for about 12 to about 24 hours.

12. The method of claim 11 wherein the hematopoietic stem cells are in contact with the YTHDF2 repressor compound for about 16 to about 20 hours.

13. The method of claim 12 wherein the hematopoietic stem cells are in contact with the YTHDF2 repressor compound for about 20 hours.

14. A method of preparing hematopoietic stem cells for homing during stem cell transplantation, comprising
a. contacting hematopoietic stem cells with an FTO expression activator compound for about 8 to about 48 hours; and
b. removing the FTO expression activator compound from contact with the hematopoietic stem cells prior to transplantation of the hematopoietic stem cells into a subject.

15. The method of claim 14 wherein the hematopoietic stem cells are harvested from embryonic cord blood.

16. The method of claim 14 wherein the FTO expression activator compound is Angiotensin II.

17. The method of claim 16 wherein the amount of Angiotensin II is about 2 μM to about 10 μM.

18. The method of claim 17 wherein the amount of Angiotensin II is about 4 μM,

19. The method of claim 14 wherein the hematopoietic stem cells are in contact with the FTO expression activator compound for about 20 to about 24 hours.

20. The method of claim 19 wherein the hematopoietic stem cells are in contact with the FTO expression activator compound for about 20 hours

21. A method of treatment of cord blood cells in preparation for homing, comprising
a. contacting cord blood cells with a YTHDF2 repressor compound for about 6 to about 48 hours; and
b. removing the YTHDF2 repressor compound from contact with the cord blood cells prior to transplantation of the cord blood cells into a subject.

22. The method of claim 21 wherein the YTHDF2 repressor compound is Raloxifene.

23. The method of claim 21 wherein the YTHDF2 repressor compound is Imatinib.

24. The method of claim 21 wherein the cord blood cells are in contact with the YTHDF2 repressor compound for about 20 to about 24 hours.
